(19) United States
(12) Patent Application Publication
(10) Pub. No.: US 2008/0210228 A1
(43) Pub. Date: Sep. 4, 2008

(54) Monodose nasal sprayer

(75) Inventor: Hipolito P. Corbacho, Green Brook, NJ (US)

Correspondence Address: Kenneth P. Glynn, 24 Mine Street, Flemington, NJ, 08822, US

(73) Assignee: Corbco, Inc.

(21) Appl. No.: 11/713,335

(22) Filed: Mar. 2, 2007

(51) Int. Cl. A61M 15/08

(52) U.S. Cl. 128/200.22

(57) ABSTRACT

The sprayer device utilizes a friction held piston that fires automatically upon full depression of the firing cap. The drive spring does not release to fire the piston until after the spring is fully compressed because the force required to overcome the frictional force between said high friction engaging surface of the piston and the piston shaft is greater than the force required to compress said spring. A user may procure a monodose of medicine by (i) unlocking a child resistant locking mechanism; (ii) applying compressive force against the firing cap so as to first compress the spring and then to push the piston out of an piston engaging friction area such that the spring causes the distal end of the piston to break the medicine container to drive medicine through the spray nozzle in a manner that is user independent as to dosage amount and dosage speed of delivery.

### BACKGROUND OF INVENTION

a. Field of Invention

The invention relates generally to nasal sprayers and, more particularly, to nasal sprayers that deliver a single dose that is user independent as to dosage amount and dosage speed of delivery to yield a consistent, predetermined dose and profile spray.

b. Description of Related Art

The following patents are representative of sprayers:

U.S. Pat. No. 7,000,806 B2 to Daniel Py et al. describes a dispenser for dispensing a fluid which includes a rigid vial that has a main fluid chamber containing a fluid, and a pump assembly that is in fluid communication with the main fluid chamber and is configured to dispense a predetermined quantity of fluid from the main fluid chamber. A flexible bladder is provided which is located within the main fluid chamber and is configured to expand to fill the ullage created within the main fluid chamber during dispensing of fluid by the pump assembly. The resilient bladder tends to force itself outwardly toward the rigid vial and, in turn, increases the pressure within the main fluid chamber in comparison to the interior of the bladder to thereby prevent the ingress of air or vapors through the bladder or otherwise into the main fluid chamber.

U.S. Pat. No. 6,997,219 to Daniel Py et al. describes a dispenser for holding multiple doses of fluids or other substances, and for dispensing the substances, which has a vial, a flexible bladder received within the vial, and a variable volume storage chamber formed between the bladder and vial. A filling valve is coupled in fluid communication with the storage chamber and defines a normally closed, fluid-tight position hermetically sealing the storage chamber from the ambient atmosphere, and an open position allowing the passage of fluid through the valve both to evacuate the storage chamber and to introduce fluid through the valve to fill the storage chamber. A pump is coupled in fluid communication with the storage chamber for pumping fluids out of the storage chamber. A dispensing valve is coupled in fluid communication with the pump and defines a normally closed, fluid-tight position preventing the passage of fluid out of the dispenser, and an open position for dispensing pumped fluid therethrough. The sealed, empty dispenser is sterilized, such as by applying gamma radiation thereto. Then, the sterilized, sealed, empty dispenser is filled with fluid by engaging the filling valve with an evacuating/dispensing member to evacuate the storage chamber, and by introducing fluid from the filling member through the open filling valve and into the storage chamber. The filling member is withdrawn from the valve, and a spring moves the valve to a closed position to hermetically seal the fluid within the dispenser.

U.S. Pat. No. 6,419,663 B2 to John E. Harrold describes the present invention mechanically propelled, liquid metered dispenser which includes a main housing with liquid storage and a liquid advancing component for either allowing liquid to flow out or assisting in the flow of liquid out of the main housing by exerting pressure, into a metered dosage dispensing chamber in response to a loading movement of a cocking mechanism. The chamber has an inlet connected to the main housing with a one-way valve to only permit flow of liquid into the chamber. The chamber also has an outlet orifice for dispensing liquid therefrom in a metered amount. The chamber has a reciprocal plunger and has a load and release component connected to it, which includes a cocking mechanism is functionally connected to the plunger so as to move in harmony therewith, or, more preferably, to move a relatively short distance relative to the distance traveled by the cocking mechanism.

U.S. Pat. No. 6,302,101 B1 to Daniel Py describes a pump type dispenser for dispensing predetermined doses of medicament in droplets or in spray form to the nasal area which incorporates a rigid vial for medicament, and expandable pouch located within the rigid vial, a nasal screen, a one-way actuation mechanism, a one-way valve mechanism in the nozzle area and a spring element, both the valve mechanism and the spring element being formed as integral portions of the pump body. The nasal screen aligns the dispenser nozzle with the nasal passage and also allows the user to discreetly hide the nasal area from the public view. The one-way valve mechanism in the nozzle area ensures a one-way movement of medicament from the dispenser, thereby preserving substantially perfect sterility of the medicament in the dispenser regardless of the environment surrounding the dispenser, without requiring the use of preservatives. The one way actuation mechanism enables the user to load and dispense a uniform quantity of medicament with a uniform actuation force and speed via a single continuous motion of the actuation mechanism upon application of a very small force on the actuation trigger mechanism by the user. By preventing the pump mechanism from being left in a loaded state for a prolonged period of time, the one-way actuation mechanism prevents the spring element from experiencing the “fatigue” phenomenon. The rigid vial/expandable-pouch combination facilitates improved long term use, as well as uniformity of dosage independent of the pump orientation.

U.S. Pat. No. 6,033,384 to Daniel Py describes a medicament-dispensing system which includes a cartridge for housing and actuating an accordion-like vial-dispenser for dispensing a calibrated amount of medicament by means of a single actuation motion which sequentially loads the vial-dispenser and dispenses the loaded medicament. The vial-dispenser has a front bellows portion, a rear bellows portion, an internal piston mechanism, a medicament storage chamber and a dosage cavity. The cartridge has a trigger mechanism which acts in concert with a notched lever and a wedge-shaped arm internally located in the cartridge to sequentially load the dosage cavity with medicament and discharge it. Depression of the trigger mechanism simultaneously extends, by means of the notched lever, the front bellows portion and compresses the rear bellows portion to load the dosage cavity with medicament. Once the notched lever has extended the front bellows portion a predetermined distance, the notched lever is disengaged form the front bellows portion by the wedge-shaped arm extending from the rear wall of the housing, thereby releasing the front and rear bellows portions, along with the internal piston mechanism, to return to original position and force the medicament from the dosage cavity via a nozzle of the vial-dispenser.

U.S. Pat. No. 5,979,711 to Karl-Heinz Fuchs et al. describes that in a device suitable for discharging media or at least one springly deformable component consists of a copolymer produced with a co-catalyst such as titanocen, more particularly an ethylene α-olefin copolymer which may also be improve as regards its resiliency by gamma irradiation. In a spiral spring produced from this or a similar plastics material axially adjacent spiral sections are directly integrally connected to each other not only via the spiral but also along the circumference of the spring.

U.S. Pat. No. 5,685,869 to Daniel Py describes an apparatus that is used to apply medicament to an eye and stores the medicament in a medicament chamber. A nozzle is coupled in fluid communication with the medicament chamber and is formed by an outer nozzle portion and an inner nozzle portion received within the outer nozzle portion. A seam is formed by the interface of the inner nozzle portion and the outer nozzle portion and is normally in a closed position to prevent the passage of medicament through the nozzle. The seam opens in response to the flow of medicament of sufficient pressure into the seam to permit the passage of medicament through the nozzle for release into the eye.

U.S. Pat. No. 5,613,957 to Daniel Py describes an apparatus that is used to apply medicament to an eye and stores the medicament in a medicament chamber. A nozzle is coupled in fluid communication with the medicament chamber and is formed by an outer nozzle portion and an inner nozzle portion received within the outer nozzle portion. A tight interface is defined between the inner nozzle portion and the outer nozzle portion and is normally in a closed position to prevent the passage of medicament through the nozzle. The interface opens in response to the flow of medicament of sufficient pressure into it to permit the passage of medicament through the nozzle for release into the eye.

U.S. Pat. No. 5,401,259 to Daniel Py describes a cartridge for actuating a piston-like or accordion-like dispenser-vial for applying a medicament to an eye. The cartridge includes a housing for holding the dispenser-vial and a telescoping cylinder for compressing the dispenser-vial in the longitudinal direction to actuate the vial. The cartridge includes a locking mechanism for locking the telescoping cylinder to restrict its movement and a lever mechanism for releasing the cylinder from the locked position so that a drop is released from the dispenser. The housing includes a finger for engaging the lower eyelid and exposing the conjunctival culde-sac.

U.S. Pat. No. 5,267,986 to Daniel Py describes a cartridge for actuating a piston-like or accordion-like dispenser-vial for applying medicament to an eye. The cartridge includes a housing for holding the dispenser-vial and a telescoping cylinder for compressing the dispenser-vial in the longitudinal direction to actuate the vial. The cartridge includes a locking mechanism for locking the telescoping cylinder to restrict its movement and a trigger mechanism for releasing the cylinder from the locked position so that a drop is released from the dispenser. The housing includes a finger for engaging the lower eyelid and exposing the conjunctival cul de sac.

U.S. Pat. No. 5,238,156 to Raimund Andris describes that in a metering and spray pump for liquid, low-viscosity and pasty substances, an elastic bellows is arranged between two plastic housing parts that are telescopingly movable relative to one another, connecting them. The bellows, acting as a discharge valve has, at one end, a valve annular wall that surrounds the generated surface of an inner annular discharge seat made in one piece with the first housing part in a sealing manner and such that it can be lifted off. As a suction valve the bellows has, at its other end, a valve annular wall which is in sealing and separable contact with the generated surface of a valve seat made in one piece with the second housing part such that it can be lifted off, and the medium to be pumped is drawn into the bellows through the valve seat. To guarantee high reliability of operation, especially good closing quality at weak valve opening forces, where the quality of closing can be tested even in the dry state, with the smallest possible number of simple and easy to assemble individual parts, the valve annular wall of the discharge valve and the valve annular wall of the suction valve, which valve annular wall is provided with a closed front wall, are each is contact with conical or hemispherical generated surfaces, wherein both valve annular walls are connected to the bellows both radially elastically and elastically movable in the axial direction.

U.S. Pat. No. 4,944,430 to Lothar Graf et al. describes a vacuum-tight and pressure-tight closed storage space in a container that is constructionally combined with a thrust piston pump projecting thereinto and a drag piston slideably guided therein and in the initial position of the thrust piston pump is hermetically sealed by an exhaust valve mechanically closed in forcibly controlled manner. The drag piston is adapted to the thrust piston pump in such a way that the storage space can be emptied substantially free with the thrust piston pump.

U.S. Pat. No. 3,934,585 to David M. Maurice describes a method and apparatus for applying therapeutic eye drops to the eye by metering a predetermined volume of fluid and rapidly applying a pressure to one end of the metered fluid for forcing the fluid from a nozzle of means defining a small passageway such as an open ended tube as a droplet having sufficient velocity to travel a generally horizontal distance in space to the eye. Unit dose application and multiple dose application are included and provision is made for preventing anticipatory blinking of the eye during self-administration.

Notwithstanding the prior art, the present invention is neither taught nor rendered obvious thereby.

### SUMMARY OF INVENTION

The present invention is a monodose nasal sprayer device that eliminates the usual uncertainties of nasal sprayers, to with, the speed of depression that affects the size and shape (profile) of the spray which in turn affects the efficacy of delivery; and the length of depression of the a typical nasal spray pump mechanism that affects the amount of dosage. These variables can have adverse affects on the proper treatment of a nasal condition and are completely eliminated by the present invention. Thus, the present invention has been developed to drive medicine through the spray nozzle in a manner that is user independent as to dosage amount and dosage speed of delivery to yield a consistent, predetermined dosage and a consistent predetermined profile spray.

The present invention includes an elongated main housing, a central member and a firing cap, as well as internal components. The main housing has a distal end adapted for partial insertion into a human nasal cavity and has a proximal end adapted to receive and hold the breakable monodose medicine package-supporting central member. The elongated main housing has a spray discharge nozzle located at its distal end, and it has a medicine-receiving chamber connected to the nozzle and biased toward the breakable monodose medicine package-supporting central member.

The central member is a breakable medicine package-supporting central member is connected to the proximal end of the elongated main housing. The connection may be permanent or removable, and it may be threaded, heat welded, snap fitted or otherwise connected and the central unit may be connect into the inside of the main housing, or vice versa. The central member has a distal end adapted to support a breakable monodose medicine package so as to be positioned at the chamber, and has a piston shaft with a piston-engaging high friction area end and a piston-releasing area distal end. A piston is located in the piston shaft, the piston having a medicine container breaking distal end, having a central area with a high friction engaging surface to engage the proximal end of the piston shaft and having an extended proximal end functionally connected to a firing cap. A firing cap is connected to the proximal end of the central member and is moveable toward the distal end of the central member by compressive force. The firing cap has a piston-receiving cavity and a drive spring located in the cavity and the extended proximal end of the piston is functionally connected to the spring.

A finger support means is located on the outside of one of the main housing and the central member. A child resistant locking mechanism is connected to at least one of the central member and the firing cap that is unlockable and, when locked, prevents movement of the firing cap toward the distal end of the central member, and permits movement of the firing cap toward the distal end of the central member when the locking member is unlocked.

An essential feature is that the force required to overcome the frictional force between the high friction engaging surface of the piston and the piston shaft is greater than the force required to compress the spring. A user may procure a monodose of medicine with the device when loaded with a breakable monodose medicine package by (i) moving the child resistant locking mechanism from its locked position to its unlocked position; (ii) placing fingers in front of the finger support means and applying compressive force against the firing cap to move the firing cap towards the central member so as to first compress the spring before moving the piston, and to move the firing cap closer toward the central member to push the piston out of the piston engaging friction area of the shaft so as to sufficiently reduce friction such that the spring automatically and with significant celerity, fires the piston and causes the distal end of the piston to puncture the breakable monodose medicine package. This automatically drives medicine therefrom to and through the spray nozzle in a manner that is user independent as to dosage amount and dosage speed of delivery to yield a consistent, predetermined speed of delivery and a predetermined profile spray.

In some preferred embodiments, the present invention monodose nasal sprayer device piston shaft and the piston have corresponding circular peripheries from a top view.

In some preferred embodiments of the present invention monodose nasal sprayer device, the distal end of the central member and the proximal end of the main housing have corresponding screw components and the central member distal end and the main housing proximal end are screwed to one another.

In some preferred embodiments of the present invention monodose nasal sprayer device, the screw components include a one way locking thread stop so that the distal end of the central member and the proximal end of the main housing cannot be unscrewed once screwed together.

In some preferred embodiments of the present invention monodose nasal sprayer device, the proximal end of the piston includes a flange that fits atop the spring and fits into the firing cap cavity.

In some preferred embodiments of the present invention monodose nasal sprayer device, the child resistant locking mechanism is a rotating locking mechanism with a first position being a lock position to inhibit advancement of the firing cap and a second position being an unlock position to permit advancement of the firing cap, the second position being located at an arc of a predetermined distance from the first position.

In some preferred embodiments of the present invention monodose nasal sprayer device, the child resistant locking mechanism includes a protrusion on the firing cap and a receiving track on the central member such that when the protrusion is positioned in alignment with the receiving track, it is in its unlock position and the firing cap may be advanced toward the central member.

In some preferred embodiments of the present invention monodose nasal sprayer device, the chamber of the main housing is a dome-shaped chamber and the distal end of the piston is similarly domed-shaped.

In some preferred embodiments of the present invention monodose nasal sprayer device, the device further includes a breakable monodose medicine package mounted on the distal end of the central member and at least partially projecting into the chamber.

In some preferred embodiments of the present invention monodose nasal sprayer device, at least one of the piston and the shaft includes at least one friction ring.

In some preferred embodiments of the present invention monodose nasal sprayer device, the distal end of the central member and the proximal end of the main housing have corresponding snap-together components and the central member distal end and the main housing proximal end are snapped into one another.

In some preferred embodiments of the present invention monodose nasal sprayer device, the device includes: a.) an elongated main housing having a distal end adapted for partial insertion into a human nasal cavity and having a proximal end adapted to receive and hold a breakable monodose medicine package-supporting central member, the elongated main housing having a spray discharge nozzle located at its distal end, the elongated main housing having medicine-receiving chamber connected to the nozzle and biased toward the breakable monodose medicine package-supporting central member; b.) the breakable medicine package-supporting central member being connected to the proximal end of the elongated main housing, the central member having a distal end adapted to support a breakable monodose medicine package so as to be positioned at said chamber, and having a piston shaft with a piston-engaging high friction area end and having a piston-releasing area distal end; c.) a piston located in the piston shaft, the piston having a medicine container breaking distal end, having a central area with a high friction engaging surface to engage the proximal end of the piston shaft and having an extended proximal end functionally connected to a firing cap; d.) a firing cap proximal connected to the proximal end of the central member and being moveable toward the distal end of the central member by compressive force, the firing cap having a piston-receiving cavity and a spring drive spring located in the cavity, the extended proximal end of the piston being functionally connected to the spring; e.) finger support means located on the outside of one of the main housing and the central member; and, f.) a child resistant locking mechanism connected to at least one of the central member and the firing cap that is unlockable and, when locked, prevents movement of the firing cap toward the distal end of the central member, and permits movement of the firing cap toward the distal end of the central member when the locking member is unlocked; g.) a post firing lock-up mechanism connected to the central member and the firing cap such that, once the device has been fired and the firing cap has been advanced forward toward the central member, this locking mechanism is activated and locks up so as to prevent movement of the firing cap away from the distal end of the central member; wherein the force required to overcome the frictional force between said high friction engaging surface of the piston and the piston shaft is greater than the force required to compress said spring; and, wherein a user may procure a monodose of medicine with the device when loaded with a breakable monodose medicine package by (i) moving the locking mechanism from its locked position to its unlocked position; (ii) placing fingers in front of the finger support means and applying compressive force against the firing cap to move the firing cap towards the central member so as to compress the spring and to move the firing cap closer toward the central member to push the piston out of the piston engaging friction area of the shaft so as to sufficiently reduce friction such that the spring automatically and with significant celerity, fires the piston and causes the distal end of the piston to puncture the breakable monodose medicine package and to drive medicine therefrom to and through the spray nozzle in a manner that is user independent as to dosage amount and dosage speed of delivery to yield a consistent, predetermined profile spray.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detail description serve to explain the principles of the invention. In the drawings:

FIG. 1 illustrates a front cut view of one preferred embodiment of a present invention monodose nasal sprayer;

FIG. 2 illustrates a side cut view of one embodiment of a breakable monodose medicine container that may be used in present invention sprayers;

FIG. 3 shows the breakable monodose medicine container shown in FIG. 2 in a collapsed state after it has been broken and the medicine has been dispensed;

FIGS. 4 and 5 illustrate side cut views of another embodiment of a present invention monodose nasal sprayer at different stages of use;

FIGS. 6, 7 and 8 illustrate top, front and bottom views of yet another embodiment of a present invention monodose nasal sprayer;

FIG. 9 shows the present invention monodose nasal sprayer of FIGS. 6, 7 and 8 in its rest position;

FIG. 10 shows the present invention monodose nasal sprayer of FIGS. 6, 7 and 8 in its fully compressed spring position (being squeezed);

FIG. 11 shows the present invention monodose nasal sprayer of FIG. 6, 7 and 8 in its fully compressed spring with advancing piston position (being squeezed);

FIG. 12 shows the present invention monodose nasal sprayer of FIGS. 6, 7 and 8 in its just fired position;

FIG. 13 is a cut bottom view of one embodiment of a rotating locking mechanism in its locked, child resistant position;

FIG. 14 is a cut bottom view of the rotating locking mechanism of FIG. 13 in its unlocked position ready for firing; and,

FIGS. 15, 16, 17 and 18 illustrate another present invention embodiment wherein they show side cut views of the sprayer at different stages using a pierceable vial for the medicine container.

### DETAILED DESCRIPTION OF THE EMBODIMENTS

Referring now to the drawings, like reference numerals designate corresponding parts throughout the several views.

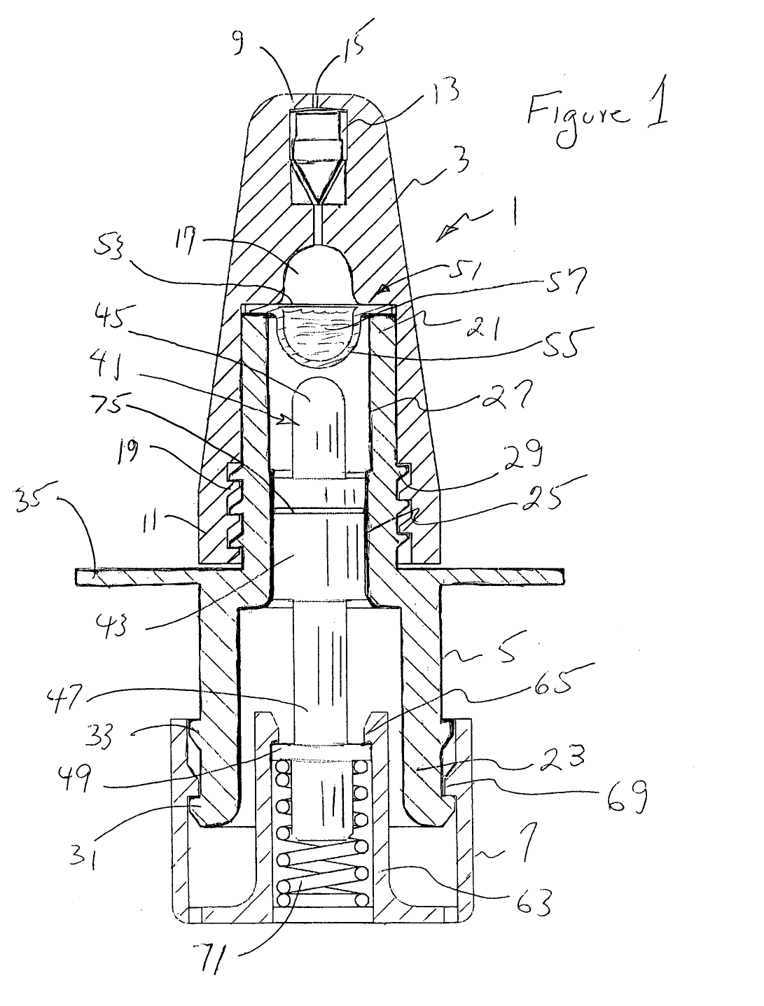

FIG. 1 illustrates a front cut view of one preferred embodiment of a present invention monodose nasal sprayer 1. It includes an elongated main housing 3 having a distal end 9 adapted for partial insertion into a human nasal cavity. Main housing 3 also has a proximal end 11 adapted to receive and hold a breakable monodose medicine package-supporting central member 5. The elongated main housing 3 has a spray discharge mechanism 13 and nozzle 15 located at its distal end 9. It also has medicine-receiving chamber 17 connected to the nozzle 15 via spray discharge mechanism 13. Chamber 17 is biased toward the breakable monodose medicine package-supporting central member 5 and is adjacent to central member 5's medicine package support, as shown.

The breakable medicine package-supporting central member 5 is connected to the proximal end 11 of the elongated main housing 3. In this embodiment, the connection is via locking threads such as thread 19 on main housing 3 and thread 29 on central member 5. The central member 5 has a distal end 21 adapted to support breakable monodose medicine package 51 so as to be positioned at (directly below) main housing chamber 17, as shown. Central member 5 also has a piston shaft 27 with a piston-engaging high friction area end 25 and has a piston-releasing area distal end 21. There is a piston 41 located in the piston shaft 27, with piston 41 having a medicine container breaking distal end 45, having a central area 43 with a high friction engaging surface to engage the proximal end of the piston shaft 25 and having an extended proximal end 47 with flange 49, functionally connected to firing cap 7. The piston 41 has one or more friction rings such as ring 75, but could have alternative or additional friction creating means, such as abraded areas, etched areas, molded three dimensional topography areas, friction creating sprays, coatings or layers, friction bands or combinations thereof. Firing cap 7 is connected to the proximal end 23 of the central member 5 and is moveable toward the distal end 21 of central member 5 by compressive force. The firing cap 7 has a piston-receiving cavity 63 and a drive spring 71 located in the cavity 63. The extended proximal end 47 of the piston 41 is functionally connected to the spring 71 as shown. Piston flange 49 is positioned under rim 65 and nests atop spring 71, as shown.

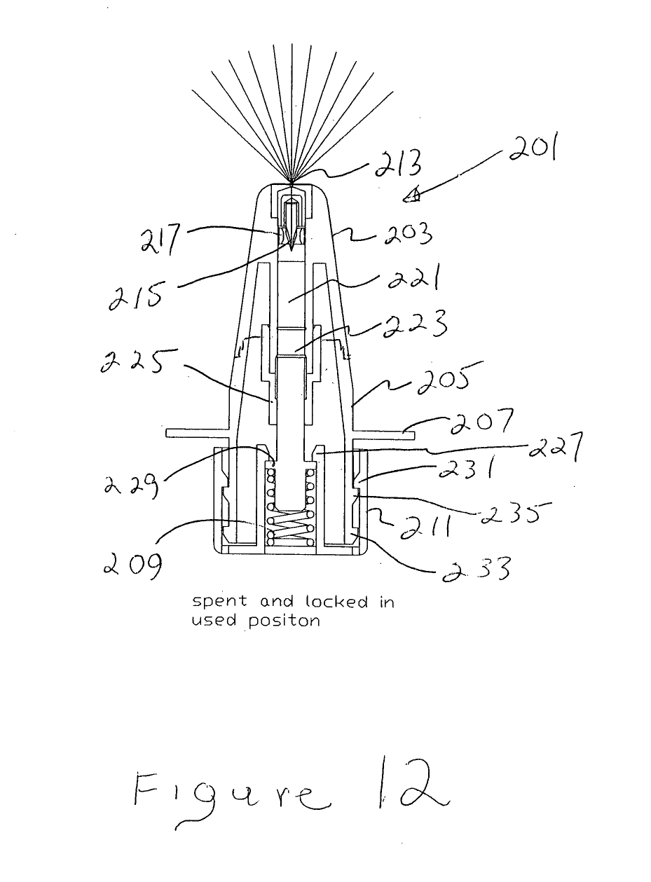

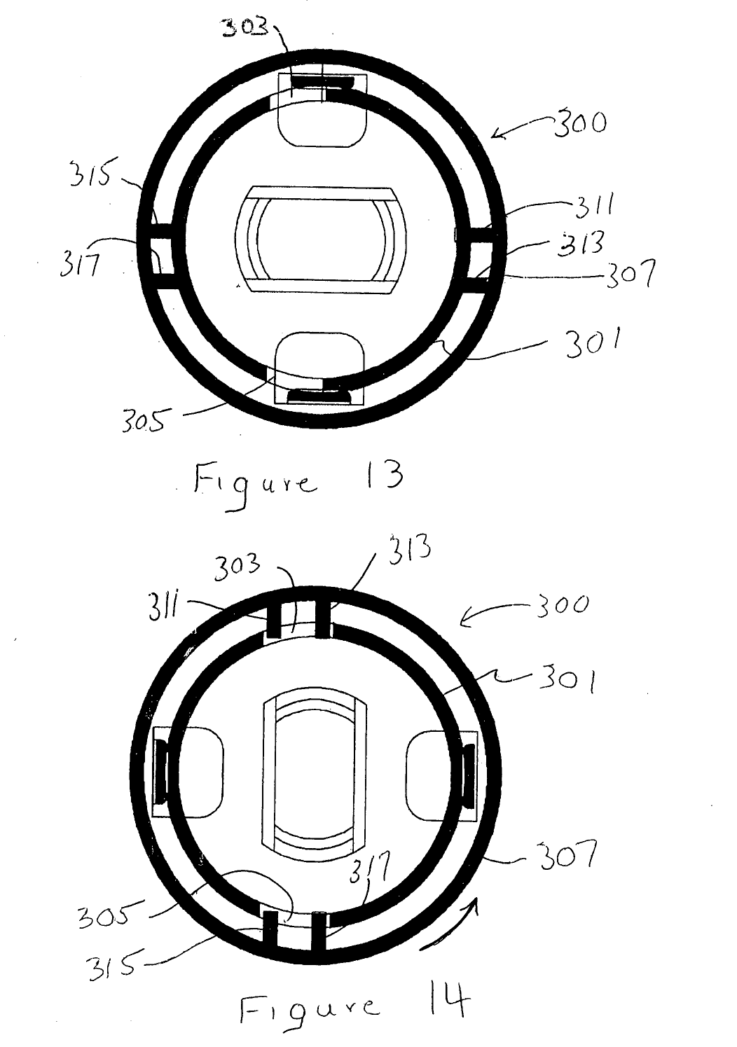

There is a finger support means 35 located on the outside of one of the main housing 3 and the central member 5. In this drawing, it is located on the outside of central member 5, but would be equally functional if located on the outside of main housing 3. A child resistant locking mechanism is connected to at least one of the central member 5 and the firing cap 7 that is unlockable and, when locked, prevents upward movement of the firing cap 7 toward the distal end 23 of the central member 5, and permits upward movement of the firing cap 7 toward the distal end 23 of the central member 5 when the locking member is unlocked. In this drawing the CR locking mechanism is hidden, but FIGS. 13 and 14 below show bottom view details of one such mechanism that could be used in this embodiment.

A post firing lock-up mechanism is connected to the central member 5 and the firing cap 7 such that, once the device has been fired and the firing cap 7 has been advanced forward (upward) toward the central member 5, this locking mechanism is activated and locks up so as to prevent movement of the firing cap 7 away from the distal end 23 of the central member 5. In this embodiment, the distal end 23 of central member 5 has two external protrusions 31 and 33 and firing cap 7 has an internal protrusion 69. (For purposes of this drawing, the protrusions are 360 degree full circle protrusions, but could be segmented or opposing pairs, or one component could be full circle and the other be segmented, e.g., protrusion 69 is fill circle and protrusions 31 and 33 are arcs.)

An essential feature of this and all present invention embodiments relates to the relative forces between the piston and the spring. Specifically, the force required to overcome the frictional force between said high friction engaging surface of the piston 41 and the piston shaft high friction area 25 is greater than the force required to compress spring 71. In this way, a user may procure a monodose of medicine with the device when loaded with a breakable monodose medicine package 51, in this case with base 53, dome 55 and medicine 57. The user accomplishes the results by (i) moving the locking mechanism from its locked position to its unlocked position; (ii) placing fingers in front of the finger support means and applying compressive force against the firing cap to move the firing cap towards the central member so as to compress the spring and to move the firing cap closer toward the central member to push the piston out of the piston engaging friction area of the shaft so as to sufficiently reduce friction such that the spring automatically and with significant celerity, fires the piston and causes the distal end of the piston to puncture the breakable monodose medicine package and to drive medicine therefrom to and through the spray nozzle in a manner that is user independent as to dosage amount and dosage speed of delivery to yield a consistent, predetermined profile spray. The post firing lock-up mechanism automatically locks the device after use so that it cannot be refired.

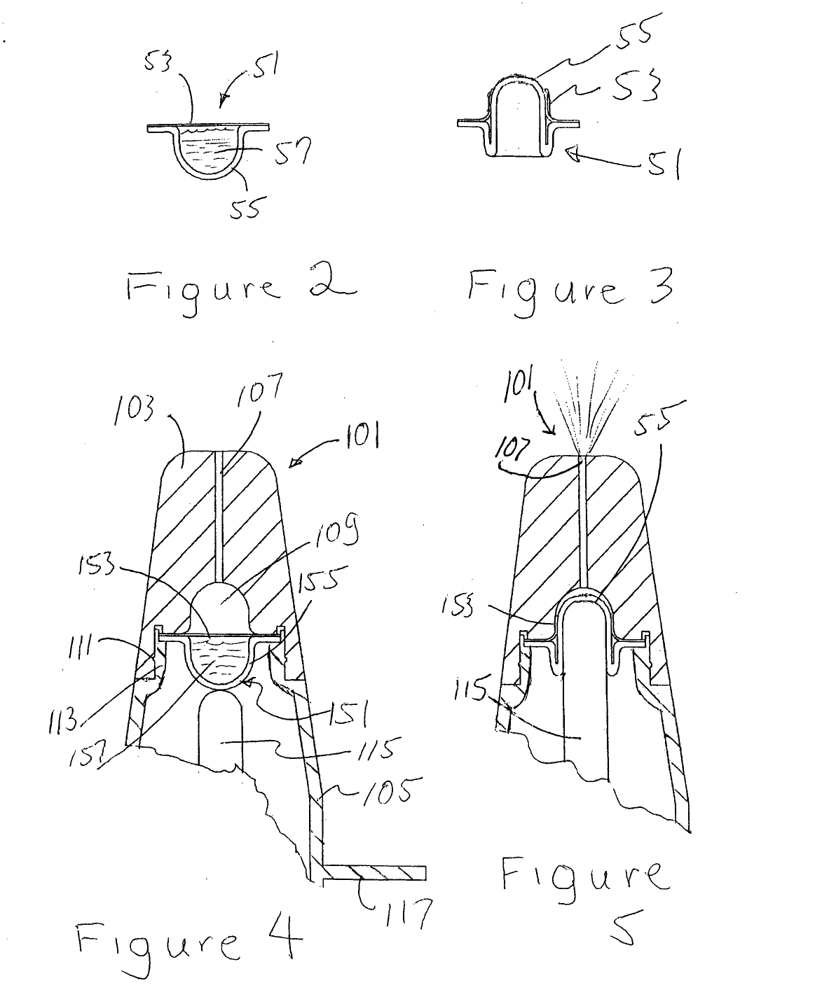

FIG. 2 illustrates a side cut view of one embodiment of a breakable monodose medicine container 51 that may is used in the present invention sprayer device described above in FIG. 1. It has a base 53 and a dome 55. The base 53 and the dome 55 may be made of the same or different materials, such as plastic, paper, foil, plastic-coated paper and combinations thereof. The most important aspects of this container 51 is that it be (1) sized and shaped for the present invention device (2) sufficiently sealed so as to not leak before use and (3) sufficiently breakable to deliver the medicine in the present invention device when struck by the piston against the chamber of the main housing. FIG. 2 shows the container 53 in its unused (unspent) form with medicine 57 of a predetermined monodosage. FIG. 3 shows the same breakable monodose medicine container 51 shown in FIG. 2 in a collapsed state after it has been broken and the medicine has been dispensed.

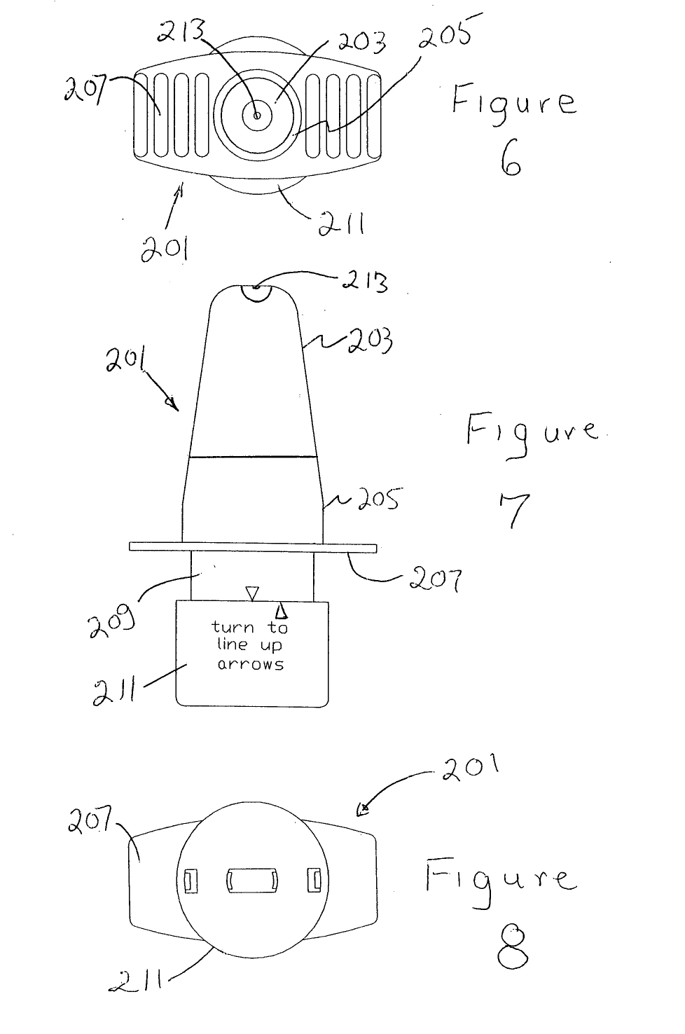

FIGS. 4 and 5 illustrate side cut views of another embodiment of a present invention monodose nasal sprayer 101 at two different stages of use. (Both FIGS. 4 and 5 are discussed together.) Sprayer 101 includes a main housing 103, a central member 105 and a firing cap (not Shown) that is the same as the one shown in FIG. 1 above and works in the same manner. These two Figures are presented to illustrate the before and after states of firing the device and discharging the medicine. Main housing 103 has a distal end with a spray discharge nozzle 107, and a proximal end with a connected chamber 109. It is snap fit connected to central member 105 at member 105's distal end 113, as shown. Central member 105 supports the medicine container that is similar to container 51 above with similar components base 153, dome 155 and medicine 157. Central member 105 also has internal components similar to the central member 5 of FIG. 1 only partially shown here with piston 115 and finger support flange 117. FIG. 4 shows the medicine 157 in its rest position before sprayer 101 is fired and FIG. 5 shows it immediately after firing, with medicine 157 dispersed through nozzle 107.

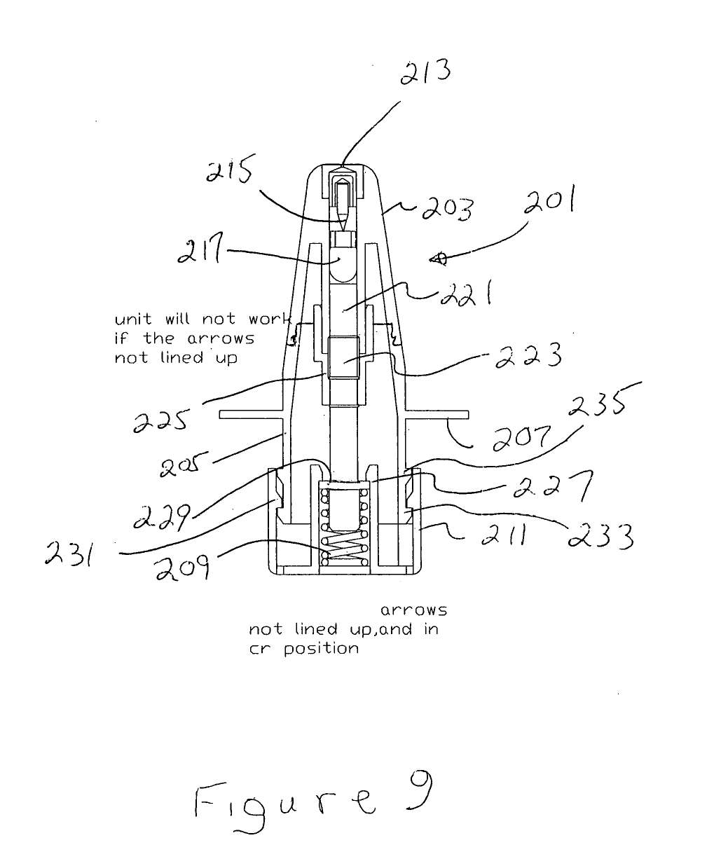

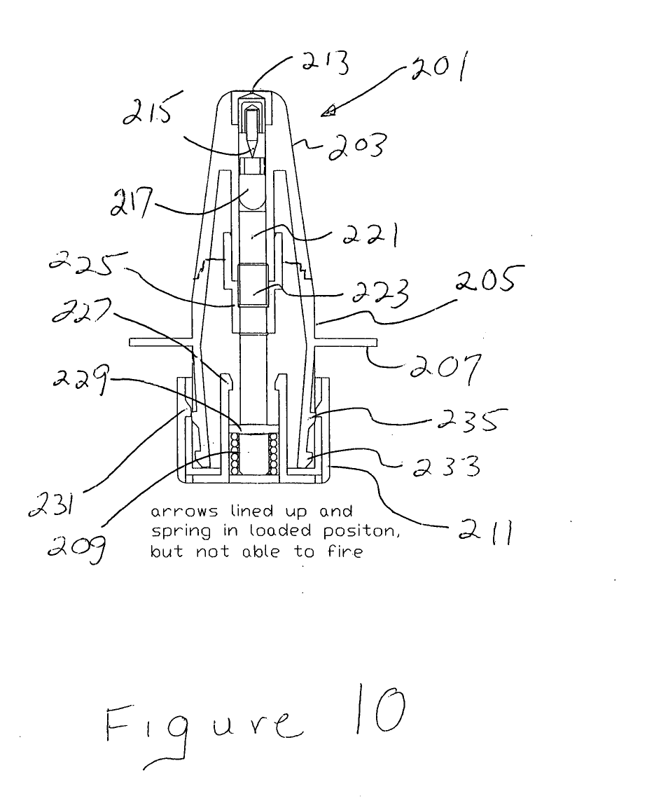

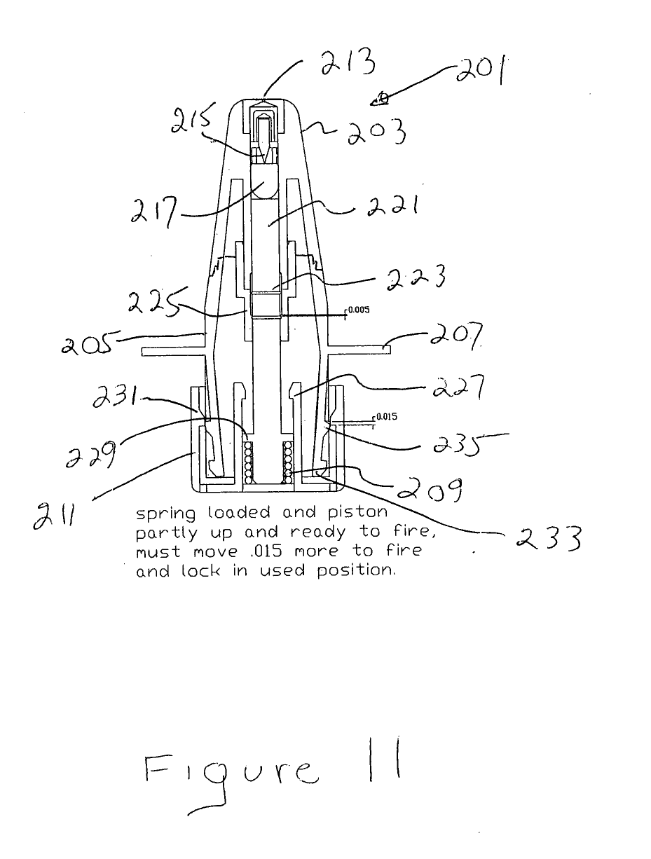

FIGS. 6, 7 and 8 illustrate top, front and bottom views of yet another embodiment of a present invention monodose nasal sprayer 201. FIG. 9 shows the present invention monodose nasal sprayer 201 of FIGS. 6, 7 and 8 in its rest position. FIG. 10 shows the present invention monodose nasal sprayer 201 of FIGS. 6, 7 and 8 in its fully compressed spring position (being squeezed). And FIG. 11 shows the present invention monodose nasal sprayer 201 of FIG. 6, 7 and 8 in its fully compressed spring with advancing piston position (being squeezed). All of these Figures have like numbered components and the Figures are discussed collectively.

Sprayer 201 includes an elongated main housing 203 having a distal end adapted for partial insertion into a human nasal cavity. Main housing 3 also has a proximal end adapted to receive and hold a breakable monodose medicine package-supporting central member 205. In this embodiment, the connection between the main housing 203 and the central member 205 is a force fit, as shown. The elongated main housing 203 has a spray discharge mechanism nozzle 213 located at its distal end and a medicine container piercing spike 215 extending downwardly therefrom into a chamber into which a medicine container is driven by the firing of the device and associated release of the piston. The medicine-receiving chamber is connected to the nozzle 213 via the spray discharge mechanism shown. The breakable monodose medicine package-supporting central member 205 has a shaft 225 and a piston 221 with the same type(s) of friction enhancing area 223 described anywhere above.

The central member 205 has a distal end adapted to support breakable monodose medicine package 217 so as to be positioned at (directly below) piercing spike 215. Central member 205 has piston shaft 225 with piston-engaging high friction area end and has a piston-releasing area distal end. Piston 221 located in the piston shaft 225, with piston 221 having a medicine container breaking distal end, having a central area with a high friction engaging surface to engage the proximal end of the piston shaft 225 and having an extended proximal end with flange 229, functionally connected to firing cap 211. The piston 221, in this embodiment, breaks the medicine container not by directly puncturing it, but by pushing it into spike 215, which breaks the container by puncture. The container 217 is a breakable plastic shell with liquid medicine therein. Firing cap 211 is connected to the proximal end of the central member 205 and is moveable toward the distal end of central member 205 by compressive force. The firing cap 211 has a piston-receiving cavity and a drive spring 209 located in the cavity. The extended proximal end of the piston 221 is functionally connected to the spring 209 as shown. Piston flange 229 is positioned under rim 227 and on spring 209, as shown.

There is a finger support means 207 located on the outside of central member 205. A child resistant locking mechanism includes the protrusions 231, 233 and 235 and operate similarly to the corresponding parts in FIG. 1. When locked, this prevents upward movement of the firing cap 211 toward the central member 205, and permits upward movement of the firing cap 211 toward the central member 205 when the locking member is unlocked.

The child resistant locking mechanism also performs as a post firing lock-up mechanism such that, once the device has been fired and the firing cap 211 has been advanced forward (upward) toward the central member 205, this locking mechanism is activated and locks up so as to prevent movement of the firing cap 211 away from the central member 205.

In FIG. 9, the arrows shown in FIG. 7 are not lined up. Thus, the device is in the children resistant mode and cannot work. In FIGS. 10 and 11, the arrows have been lined up and the user has pushed the firing cap 211 toward the central member 205 to fully compress the spring 209, but not yet far enough to release the piston 221. In FIG. 12, the firing cap 211 has been advanced an additional 0.015 inches to pop the piston 221, pierce the container 217 and spray the medicine, as shown. It operates to drive medicine therefrom to and through the spray nozzle in a manner that is user independent as to dosage amount and dosage speed of delivery to yield a consistent, predetermined rate of dispensing and profile spray. The post firing lock-up mechanism automatically locks the device after use so that it cannot be refired.

FIG. 13 is a cut bottom view of one embodiment of a rotating locking mechanism 300 in its locked, child resistant position. It shows a bottom view of a central member 301 with guide slots 3053 and 305, and of a firing cap 307 with inwardly projecting protrusions 311, 313, 315 and 317. In this FIG. 13, the guide slots and protrusions are not aligned and thus the firing cap 307 cannot be advanced. In FIG. 14, a cut bottom view of the rotating locking mechanism of FIG. 13 in its unlocked position ready for firing, they are aligned and the firing cap 307 may be advanced for firing.

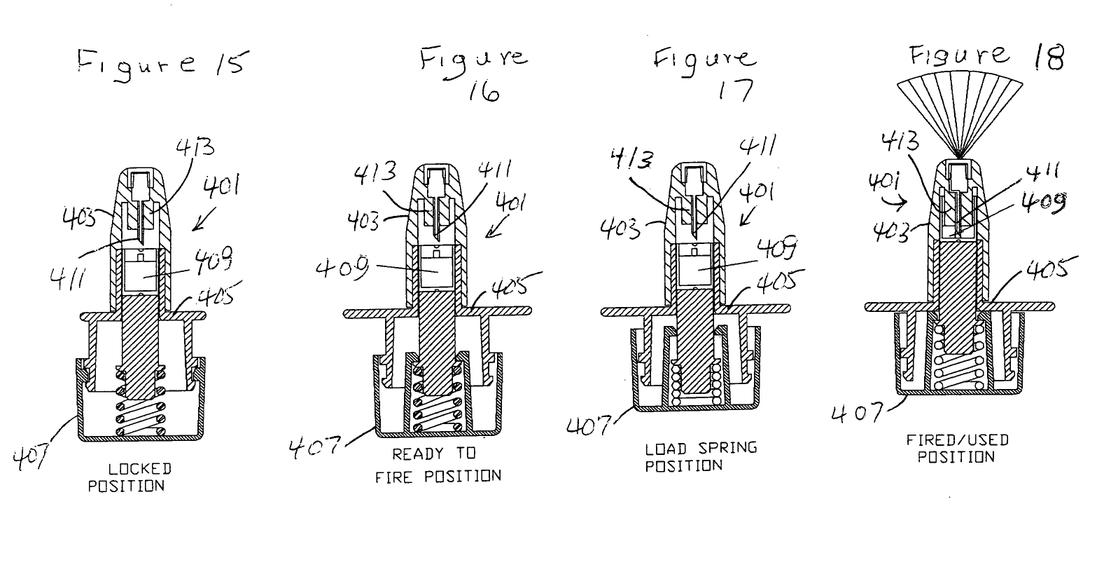

FIGS. 15, 16, 17 and 18 illustrate another present invention embodiment sprayer 401 wherein they show side cut views of the sprayer 401 at different stages using a pierceable vial 409 for the medicine container. In all of these Figures, sprayer 401 has the three feature components, namely, main housing 403, central member 405 and firing cap 407. The internal aspects are similar to and operate in the same manner as those described in FIGS. 9 through 12 above. In this device, the medicine container is pierceable vial 409 and the spike 411 is also the outlet line to the exit nozzle. As can be seen in these Figures, when the piston strikes the vial 409, spike 411 pierces the vial membrane or otherwise breakable top, and then block 413 enters the vial 409 to displace the medicine, which rapidly exits as a spray through the nozzle.

Although particular embodiments of the invention have been described in detail herein with reference to the accompanying drawings, it is to be understood that the invention is not limited to those particular embodiments, and that various changes and modifications may be effected therein by one skilled in the art without departing from the scope or spirit of the invention as defined in the appended claims.

What is claimed is:

1. A monodose nasal sprayer device, which comprises:
a.) an elongated main housing having a distal end adapted for partial insertion into a human nasal cavity and having a proximal end adapted to receive and hold a breakable monodose medicine package-supporting central member, said elongated main housing having a spray discharge nozzle located at its distal end, said elongated main housing having medicine-receiving chamber connected to said nozzle and biased toward said breakable monodose medicine package-supporting central member;
b.) said breakable medicine package-supporting central member being connected to said proximal end of said elongated main housing, said central member having a distal end adapted to support a breakable monodose medicine package so as to be positioned at said chamber, and having a piston shaft with a piston-engaging high friction area end and having a piston-releasing area distal end;
c.) a piston located in said piston shaft, said piston having a medicine container breaking distal end, having a central area with a high friction engaging surface to engage said proximal end of said piston shaft and having an extended proximal end functionally connected to a firing cap;
d.) a firing cap connected to said proximal end of said central member and being moveable toward said distal end of said central member by compressive force, said firing cap having a piston-receiving cavity and a drive spring located in said cavity, said extended proximal end of said piston being functionally connected to said spring;
e.) finger support means located on the outside of one of said main housing and said central member; and,
f.) a child resistant locking mechanism connected to at least one of said central member and said firing cap that is unlockable and, when locked, prevents movement of said firing cap toward said distal end of said central member, and permits movement of said firing cap toward said distal end of said central member when said locking member is unlocked;
wherein the force required to overcome the frictional force between said high friction engaging surface of said piston and said piston shaft is greater than the force required to compress said spring; and,
wherein a user may procure a monodose of medicine with said device when loaded with a breakable monodose medicine package by (i) moving said locking mechanism from its locked position to its unlocked position; (ii) placing fingers in front of the finger support means and applying compressive force against said firing cap to move said firing cap towards said central member so as to compress said spring and to move said firing cap closer toward said central member to push said piston out of said piston engaging friction area of said shaft so as to sufficiently reduce friction such that said spring automatically and with significant celerity, fires said piston and causes said distal end of said piston to puncture said breakable monodose medicine package and to drive medicine therefrom to and through said spray nozzle in a manner that is user independent as to dosage amount and dosage speed of delivery to yield a consistent, predetermined profile spray.

2. The monodose nasal sprayer device of claim 1 wherein said piston shaft and said piston have corresponding circular peripheries from a top view.

3. The monodose nasal sprayer device of claim 1 wherein said distal end of said central member and said proximal end of said main housing have corresponding screw components and said central member distal end and said main housing proximal end are screwed to one another.

4. The monodose nasal sprayer device of claim 3 wherein said screw components include a one way locking thread stop so that said distal end of said central member and said proximal end of said main housing cannot be unscrewed once screwed together.

5. The monodose nasal sprayer device of claim 1 wherein said piston proximal end of said piston includes a flange that fits atop said spring and fits into said firing cap cavity.

6. The monodose nasal sprayer device of claim 1 wherein said locking mechanism is a rotating locking mechanism with a first position being a lock position to inhibit advancement of said firing cap and a second position being an unlock position to permit advancement of said firing cap, said second position being located at an arc of a predetermined distance from said first position.

7. The monodose nasal sprayer device of claim 6 wherein said locking mechanism includes a protrusion on said firing cap and a receiving track on said central member such that when said protrusion is positioned in alignment with said receiving track, it is in its unlock position.

8. The monodose nasal sprayer device of claim 1 wherein said chamber of said main housing is a dome-shaped chamber and said distal end of said piston is similarly domed-shaped.

9. The monodose nasal sprayer device of claim 1 wherein said device further includes a breakable monodose medicine package mounted on said distal end of said central member and at least partially projecting into said chamber.

10. The monodose nasal sprayer device of claim 1 wherein at least one of said piston and said shaft includes at least one friction ring.

11. The monodose nasal sprayer device of claim 1 wherein said distal end of said central member and said proximal end of said main housing have corresponding snap-together components and said central member distal end and said main housing proximal end are snapped into one another.

12. A monodose nasal sprayer device, which comprises:
a.) an elongated main housing having a distal end adapted for partial insertion into a human nasal cavity and having a proximal end adapted to receive and hold a breakable monodose medicine package-supporting central member, said elongated main housing having a spray discharge nozzle located at its distal end, said elongated main housing having medicine-receiving chamber connected to said nozzle and biased toward said breakable monodose medicine package-supporting central member;
b.) said breakable medicine package-supporting central member being connected to said proximal end of said elongated main housing, said central member having a distal end adapted to support a breakable monodose medicine package so as to be positioned at said chamber, and having a piston shaft with a piston-engaging high friction area end and having a piston-releasing area distal end;
c.) a piston located in said piston shaft, said piston having a medicine container breaking distal end, having a central area with a high friction engaging surface to engage said proximal end of said piston shaft and having an extended proximal end functionally connected to a firing cap;
d.) a firing cap connected to said proximal end of said central member and being moveable toward said distal end of said central member by compressive force, said firing cap having a piston-receiving cavity and a drive spring located in said cavity, said extended proximal end of said piston being functionally connected to said spring;
e.) finger support means located on the outside of one of said main housing and said central member; and,
f.) a child resistant locking mechanism connected to at least one of said central member and said firing cap that is unlockable and, when locked, prevents movement of said firing cap toward said distal end of said central member, and permits movement of said firing cap toward said distal end of said central member when said locking member is unlocked;
g.) a post firing lock-up mechanism connected to said central member and said firing cap such that, once said device has been fired and said firing cap has been advanced forward toward said central member, this mechanism is activated and locks up so as to prevent movement of said firing cap away from said distal end of said central member;
wherein the force required to overcome the frictional force between said high friction engaging surface of said piston and said piston shaft is greater than the force required to compress said spring; and,
wherein a user may procure a monodose of medicine with said device when loaded with a breakable monodose medicine package by (i) moving said locking mechanism from its locked position to its unlocked position; (ii) placing fingers in front of the finger support means and applying compressive force against said firing cap to move said firing cap towards said central member so as to compress said spring and to move said firing cap closer toward said central member to push said piston out of said piston engaging friction area of said shaft so as to sufficiently reduce friction such that said spring automatically and with significant celerity, fires said piston and causes said distal end of said piston to puncture said breakable monodose medicine package and to drive medicine therefrom to and through said spray nozzle in a manner that is user independent as to dosage amount and dosage speed of delivery to yield a consistent, predetermined profile spray.

13. The monodose nasal sprayer device of claim 12 wherein said piston shaft and said piston have corresponding circular peripheries from a top view.

14. The monodose nasal sprayer device of claim 12 wherein said distal end of said central member and said proximal end of said main housing have corresponding screw components and said central member distal end and said main housing proximal end are screwed to one another.

15. The monodose nasal sprayer device of claim 14 wherein said screw components include a one way locking thread stop so that said distal end of said central member and said proximal end of said main housing cannot be unscrewed once screwed together.

16. The monodose nasal sprayer device of claim 12 wherein said piston proximal end of said piston includes a flange that fits atop said spring and fits into said firing cap cavity.

17. The monodose nasal sprayer device of claim 12 wherein said locking mechanism is a rotating locking mechanism with a first position being a lock position to inhibit advancement of said firing cap and a second position being an unlock position to permit advancement of said firing cap, said second position being located at an arc of a predetermined distance from said first position.

18. The monodose nasal sprayer device of claim 17 wherein said locking mechanism includes a protrusion on said firing cap and a receiving track on said central member such that when said protrusion is positioned in alignment with said receiving track, it is in its unlock position.

19. The monodose nasal sprayer device of claim 12 wherein said chamber of said main housing is a dome-shaped chamber and said distal end of said piston is similarly domed-shaped.

20. The monodose nasal sprayer device of claim 12 wherein said device further includes a breakable monodose medicine package mounted on said distal end of said central member and at least partially projecting into said chamber.

21. The monodose nasal sprayer device of claim 12 wherein at least one of said piston and said shaft includes at least one friction ring.

22. The monodose nasal sprayer device of claim 12 wherein said distal end of said central member and said proximal end of said main housing have corresponding snap-together components and said central member distal end and said main housing proximal end are snapped into one another.
